(19) United States
(12) Patent Application Publication
(10) Pub. No.: US 2025/0212920 A1
(43) Pub. Date: Jul. 3, 2025

(54) METHODS AND COMPOSITIONS FOR THE PRODUCTION OF CELL-BASED MEAT PRODUCTS

(71) Applicant: Novel Farms, Inc., Berkeley, CA (US)

(72) Inventors: Michelle Seiko Lu, Berkeley, CA (US); Maria Nieves Martinez Marshall, Berkeley, CA (US)

(73) Assignee: Novel Farms, Inc., Berkeley, CA (US)

(21) Appl. No.: 18/727,833

(22) PCT Filed: Jan. 24, 2023
(86) PCT No.: PCT/US2023/011442

§ 371 Date: Jul. 10, 2024

Related U.S. Application Data
(60) Provisional application No. 63/304,374, filed on Jan. 28, 2022.

(51) Int. Cl. A23L 13/00; A23L 31/10; C12N 5/077

(52) U.S. Cl. CPC A23L 13/00; A23L 31/10; C12N 5/0658; C12N 2513/00; C12N 2533/52; C12N 2533/54; C12N 2533/92

(57) ABSTRACT

Disclosed herein are methods and compositions for the production of cell-based meat products with an enhanced uniformity of structure. In particular, the technology of the disclosure relates to filamentous fungus-yeast biocapsules and their use in methods of manufacturing cell-based meat products.

### CROSS-REFERENCE TO RELATED APPLICATIONS

The present application claims priority to U.S. Provisional Patent Application No. 63/304,374, filed on Jan. 28, 2022, the contents of which are hereby incorporated by reference in their entirety.

### TECHNICAL FIELD

The present technology relates to methods and compositions for the production of cell-based meat products with an enhanced uniformity of structure. In particular, the technology relates to the generation of filamentous fungus-yeast biocapsules and their use in methods of manufacturing cell-based meat products.

### BACKGROUND

The following description is provided to assist the understanding of the reader. None of the information provided or references cited is admitted to be prior art to the compositions and methods disclosed herein.

Animal farming has a profound negative impact on the environment, public health, and animal welfare. It is estimated that approximately 30% of the earth's surface and 70% of all arable land is dedicated to the farming of animals, and that livestock account for 20% of total terrestrial biomass. In addition to staggering land consumption, animal farming consumes an estimated 8% of the total freshwater supply of the earth. Finally, the animal farming industry is responsible for 18% of global greenhouse gas (GHG) emissions and is the largest threat to global biodiversity.

Animal farming practices and poor animal welfare conditions are causes of foodborne illnesses. Many pathogenic viruses originate from livestock, including Swine flu and bird flu. Additionally, poor hygiene practices of handling poultry products and raw pork promote the spread of E. coli, Salmonella, Campylobacter, and parasites. Furthermore, 70% of all antibiotics used in the United States are used on farm animals, and this overuse of antibiotics is a primary cause in the emergence of antibiotic-resistant bacteria. In the United States alone, these antibiotic-resistant bacteria have created an economic burden of approximately $55 billion and has overwhelmed the healthcare system with approximately 2,000,000 infections, 250,000 hospitalizations, and a minimum of 23,000 deaths per year. Finally, in addition to posing public health threats, the animal farming industry is rife with unscrupulous animal farming practices and blatant disregard of animal welfare.

An emerging industry poised to combat the negative impacts of the animal farming industry is the cell-based meat (also known as clean meat, engineered meat, cultured meat, lab-grown meat, and cultivated meat) industry. The cell-based meat industry is estimated to decrease 7-45% energy use, 78-96% greenhouse gases, 99% land use, and 82-96% water use. Additionally, the sterile production environments of cell-based meats eliminates the need for antibiotics use and will thus prevent the emergence of antibiotic-resistant bacteria.

Current cell-based meat technology heavily relies on satellite cell culture, a technique wherein animal cells are grown in a liquid medium suspension and aggregated onto microcarriers. These cell-based meat products resemble mince meat and are devoid of the shape and texture of natural animal meat, whose overall structure is derived from biomaterial scaffolds, such as the extracellular matrix (ECM). The ECM is composed of oligosaccharides and glycoproteins that impart the biochemical and biomechanical properties of natural meat. In addition to imparting structure, the ECM contains various matrix proteins that promote cell attachment, proliferation, and dictate the overall behavior and composition of the final tissue. Thus, biomaterial scaffolds such as the ECM are important for the generation of cell-based meats.

Current methods to generate ECM and similar biomaterial scaffolds are inefficient and costly. Commercially available ECM, such as Matrigel™, is harvested from mouse sarcoma. The production of Matrigel™ is an expensive and inefficient process that requires mouse cancer cell lines and a biosafety laboratory. Non-ECM scaffolds can be made from natural biomaterials such as alginate, silk, and chitosan as well as synthetic biomaterials such as polyethylene glycol, polyglycolic acid, and polyacrylamide. However, these natural biomaterial substrates must further be functionalized with purified matrix proteins to generate a biomaterial scaffold suitable for the growth of animal cells. The purification of matrix proteins is time-consuming and costly. Furthermore, functionalizing biomaterial substrates with purified matrix proteins involves costly and time-consuming techniques such as ion beam deposition and plasma treatment. Accordingly, there is a need to develop new approaches for producing inexpensive, robust, customizable, edible biomaterial scaffolds that comprise polysaccharide-based structural elements and/or proteins for the growth and proliferation of any given animal cell type into any desired tissue.

### SUMMARY

In one aspect, the disclosure of the present technology provides a biocapsule for use in production of a cell-based meat product, wherein the biocapsule comprises a biomaterial scaffold comprising (i) a filamentous fungus, and (ii) a plurality of yeast cells.

In some embodiments, the filamentous fungus comprises mycelia.

In some embodiments, the plurality of yeast cells are genetically engineered to express one or more heterologous proteins. In some embodiments the one or more heterologous proteins are selected from one or more extracellular matrix (ECM) proteins, growth factor proteins, cell adhesion proteins, cell surface proteins, cell signaling proteins, protein motifs (synthetic or naturally-occurring), flavor proteins, or any combination thereof.

In some embodiments, the filamentous fungus comprises one or more strains belonging to a division selected from the group consisting of Ascomycota, Basidiomycota, and Zygomycota (Mucoromycota and Zoopagomycota).

In some embodiments, the filamentous fungus strain is from the Ascomycota division and comprises one or more strains belonging to a genus selected from the group consisting of Aspergillus, Penicillium, and Neurospora. In some embodiments, the filamentous fungus is from the Aspergillus genus and comprises one or more strains of Aspergillus oryzae. In some embodiments, the filamentous fungus is from the Neurospora genus and comprises one or more strains of Neurospora crassa.

In some embodiments, the filamentous fungus strain is a strain of Rhizopus oryzae or Rhizopus oligosporus from the Mucoromycota division.

In some embodiments, the plurality of yeast cells comprise one or more strains selected from the group consisting of Saccharomyces cerevisiae, Saccharomyces bayanous, Saccharomyces capensis, Schizosaccharomyces pombe, and Pichia pastoris. In some embodiments, the plurality of yeast cells comprise one or more strains of Saccharomyces cerevisiae.

In some embodiments, the one or more heterologous proteins is a chordate ECM protein, growth factor protein, cell adhesion protein, cell signaling protein, protein motif (synthetic or naturally-occurring), flavor protein, cell surface protein, or any combination thereof. In some embodiments, the heterologous chordate protein is a mammalian ECM protein, growth factor protein, adhesion molecule protein, cell signaling protein, protein motif (synthetic or naturally-occurring), flavor protein, cell surface protein, or any combination thereof.

In some embodiments, the one or more heterologous proteins comprise one or more ECM proteins selected from the group consisting of: collagen, laminin, fibronectin, and RGD motif peptides.

In some embodiments, the one or more heterologous proteins are expressed solubly, targeted to a specific subcellular localization, or secreted.

In some embodiments, the plurality of yeast cells are fixed or decellularized.

In one aspect, the disclosure of the present technology provides a cell-based meat product comprising: (i) an edible biomaterial scaffold comprising: (a) a filamentous fungus, and (b) a plurality of yeast cells; and (ii) a plurality of one or more cell types.

In some embodiments, the plurality of one or more cells types comprises one or more cell types selected from the group consisting of: myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, embryonic stem cells, mesenchymal stem cells, and any combination thereof.

In some embodiments, the plurality of yeast cells are genetically engineered to express one or more heterologous proteins.

In some embodiments, the plurality of one or more cell types is a chordate cell. In some embodiments, the chordate cell is a mammalian cell.

In some embodiments, the chordate cell is a porcine cell, a bovine cell, or a poultry cell.

In some embodiments, the filamentous fungus comprises mycelia.

In some embodiments, the one or more heterologous proteins are selected from one or more extracellular matrix (ECM) proteins, growth factor proteins, cell adhesion proteins, cell surface proteins, cell signaling proteins, protein motifs (synthetic or naturally-occurring), flavor proteins, or any combination thereof.

In some embodiments, the filamentous fungus comprises one or more strains belonging to a division selected from the group consisting of Ascomycota, Basidiomycota, and Zygomycota (Mucoromycota and Zoopagomycota).

In some embodiments, the filamentous fungus strain is from the Ascomycota division and comprises one or more strains belonging to a genus selected from the group consisting of Aspergillus, Penicillium, and Neurospora. In some embodiments, the filamentous fungus is from the Aspergillus genus and comprises one or more strains of Aspergillus oryzae. In some embodiments, the filamentous fungus is from the Neurospora genus and comprises one or more strains of Neurospora crassa.

In some embodiments, the filamentous fungus strain is a strain of Rhizopus oryzae or Rhizopus oligosporus from the Mucoromycota division.

In some embodiments, the plurality of yeast cells comprise one or more strains selected from the group consisting of Saccharomyces cerevisiae, Saccharomyces bayanous, Saccharomyces capensis, Schizosaccharomyces pombe, and Pichia pastoris. In some embodiments, the plurality of yeast cells comprise one or more strains of Saccharomyces cerevisiae.

In some embodiments, the one or more heterologous proteins is a chordate ECM protein, growth factor protein, cell adhesion protein, cell signaling protein, protein motif (synthetic or naturally-occurring), flavor protein, or cell surface protein. In some embodiments, the heterologous chordate protein is a mammalian ECM protein, growth factor protein, adhesion molecule protein, cell signaling protein, protein motif (synthetic or naturally-occurring), flavor protein, or cell surface protein.

In some embodiments, the one or more heterologous proteins comprise one or more ECM proteins selected from the group consisting of: collagen, laminin, fibronectin, and RGD motif peptides.

In some embodiments, the one or more heterologous proteins are expressed solubly, targeted to a specific subcellular localization, or secreted.

In some embodiments, the plurality of yeast cells are fixed or decellularized.

In one aspect, the disclosure of the present technology provides a method for producing a filamentous fungus-yeast biocapsule comprising: adding to a filamentous fungus culture comprising mycelia a plurality of yeast cells to form a filamentous fungus-yeast preparation, and culturing the preparation under conditions to induce spontaneous co-immobilization of the filamentous fungus and yeast to form a filamentous fungus-yeast biocapsule.

In some embodiments, the conditions to induce co-immobilization of the filamentous fungus and yeast comprises the use of a first culture medium, and a second culture medium.

In some embodiments, the plurality of yeast cells are genetically engineered to express one or more heterologous proteins.

In some embodiments, the first culture medium comprises a carbon source usable by the filamentous fungus and not usable by the yeast, and wherein the carbon source is selected from the group consisting of: gluconic acid, starch, cellulose, inulin, and other similar colloidal molecules.

In some embodiments, the second culture medium comprises a preferred carbon source for the yeast that is also usable by the filamentous fungus.

In some embodiments, the method further comprises contacting the filamentous fungus with an enzyme to modify one or more of the density, porosity, or texture of the filamentous fungus. In some embodiments, the enzyme is selected from the group consisting of: zymolase, lyticase, and glucalase.

In some embodiments, the method further comprises fixing or decellularizing the yeast cells.

In some embodiments, the filamentous fungus-yeast biocapsule is used as an edible biomaterial scaffold for cell-based meat production.

In some embodiments, the method further comprises shaping the filamentous fungus-yeast biocapsule. In some embodiments, wherein the shape of the filamentous fungus-yeast biocapsule resembles the shape of a three-dimensional meat product.

In some embodiments, the method further comprises: seeding the filamentous fungus-yeast biocapsule with a plurality of one or more cell types selected from the group consisting of: myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, embryonic stem cells, mesenchymal stem cells, and any combination thereof, to form a seeded biocapsule; and culturing the seeded biocapsule under conditions effective to induce differentiation of the plurality of one or more cell types to produce an edible cell-based meat product.

In some embodiments, the plurality of one or more cell types is a chordate cell.

In some embodiments, the chordate cell is a mammalian cell.

In some embodiments, the chordate cell is a porcine cell, a bovine cell, or a poultry cell.

In some embodiments, the one or more heterologous proteins are selected from one or more extracellular matrix (ECM) proteins, growth factor proteins, cell adhesion proteins, cell surface proteins, cell signaling proteins, protein motifs (synthetic or naturally-occurring), flavor proteins, or any combination thereof.

In some embodiments, the filamentous fungus comprises one or more strains belonging to a division selected from the group consisting of Ascomycota, Basidiomycota, and Zygomycota (Mucoromycota and Zoopagomycota).

In some embodiments, the filamentous fungus strain is from the Ascomycota division and comprises one or more strains belonging to a genus selected from the group consisting of Aspergillus, Penicillium, and Neurospora. In some embodiments, the filamentous fungus is from the Aspergillus genus and comprises one or more strains of Aspergillus oryzae. In some embodiments, the filamentous fungus is from the Neurospora genus and comprises one or more strains of Neurospora crassa.

In some embodiments, the filamentous fungus strain is a strain of Rhizopus oryzae or Rhizopus oligosporus from the Mucoromycota division.

In some embodiments, the plurality of yeast cells comprise one or more strains selected from the group consisting of Saccharomyces cerevisiae, Saccharomyces bayanous, Saccharomyces capensis, Schizosaccharomyces pombe, and Pichia pastoris. In some embodiments, the plurality of yeast cells comprise one or more strains of Saccharomyces cerevisiae.

In some embodiments, the one or more heterologous proteins is a chordate ECM protein, growth factor protein, cell adhesion protein, cell signaling protein, peptide motif (synthetic or naturally-occurring), flavor protein, or cell surface protein. In some embodiments, the heterologous chordate protein is a mammalian ECM protein, growth factor protein, adhesion molecule protein, cell signaling protein, peptide motif (synthetic or naturally-occurring), flavor protein or cell surface protein.

In some embodiments, the one or more heterologous proteins comprise one or more ECM proteins selected from the group consisting of: collagen, laminin, fibronectin, and RGD motif peptides.

In some embodiments, the one or more heterologous proteins are expressed solubly, targeted to a specific subcellular localization, or secreted.

### BRIEF DESCRIPTION OF THE DRAWINGS

FIGS. 1A-1C are images showing the co-immobilization of yeast (Saccharomyces cerevisiae) and koji (Aspergillus oryzae) mycelium. FIG. 1A is an image showing yeast reporter strain expressing cytoplasmic GFP grown with koji under conditions that promote the adsorption of the yeast cell wall to the koji hyphal structures comprising the mycelium. After extensive washing, a subpopulation of the yeast still remains associated with the hyphae of the koji mycelium. The co-immobilized yeast and koji structures can be grown into flat sheets (FIG. 1B) or as spheres (FIG. 1C).

FIG. 2 is an image showing myoblasts adhere to scaffolds composed of a symbiotic growth of Aspergillus oryzae and Saccharomyces cerevisiae. Quail myoblasts (QM7 cell line) were seeded onto flat sheet-like scaffolds composed of a symbiotic growth S. cerevisiae and A. oryzae grown in the manner described in Example 7 (Scaffold generation—for flat sheets). Cell-laden scaffolds were fixed and stained for actin (visualized with 543-phalloidin), which stains animal cells.

FIGS. 3A and 3B are images showing cell-laden scaffolds assembled into structured cultivated meat products. FIG. 3A is an image showing cell-laden scaffolds that were harvested, dyed with red coloring, and crosslinked with microbial transglutaminase to produce a “raw” cultivated meat product. FIG. 3B is an image of the cultivated meat product cooked in avocado oil.

FIG. 4 is a plasmid map of a S. cerevisiae cytoplasmic expression plasmid that encodes a bovine COL1A1 45 kDa fragment. A 45 kDa fragment of the bovine (Bos taurus) COL1A1 fragment was appended with a 6× HIS epitope (“6× HIS-Bt_COL1A1^{45 kDa}”) and terminated with a 3′ UTR from the yeast CYC1 gene (“3′UTR_CYC1”). Expression is driven by the yeast GPD promoter (“P_GPD”). The plasmid contains a kanamycin resistance marker for E. coli selection (“Kan_R”), an origin of replication from the pUC plasmid for E. coli propagation (“Ori_pUC”), a LEU2 auxotrophic marker for yeast selection (“LEU2”), and a 2 um element for yeast propagation (“2 um”).

FIGS. 5A-5C are images showing myoblasts adhered to scaffolds composed of a symbiotic growth of Aspergillus oryzare and Saccharomyces cerevisiae engineered to express bovine collagen COL1A1. Quail myoblasts (QM7 cell line) were seeded onto flat sheet-like scaffolds composed of a symbiotic growth S. cerevisiae expressing a 45 kDa fragment of bovine COL1A1 and A. oryzae grown in the manner described in Example 8 (Scaffold generation—for flat sheets). Cell-laden scaffolds were fixed and stained for actin (visualized with 543-phalloidin; FIG. 5A) and nuclei (visualized with DAPI; FIG. 5B), and co-visualized (FIG. 5C).

FIG. 6A-6C are images showing cell-laden, genetically engineered scaffolds assembled into structured cultivated meat products. FIG. 6A is a top view of cell-laden scaffolds, with S. cerevisiae genetically engineered to express cytoplasmic collagen, that were harvested, and crosslinked with transglutaminase to produce a “raw” quail cell (QM7) cultivated meat product. FIG. 6B is a side view image of the cultivated meat product in FIG. 6A. FIG. 6C is an image of the cultivated meat product cooked in avocado oil.

FIG. 7 is a plasmid map of a S. cerevisiae secretion expression plasmid that encodes a bovine COL1A1 45 kDa fragment. A 45 kDa fragment of the bovine (Bos taurus) COL1A1 fragment (“Bt_COL1A1^{45 kDa}”) was fused to the 3′end of a Mating factor alpha leader sequence fused to a 6× histidine epitope (“MFa-6× HIS”) followed by a thrombin cleavage site (“thrombin cleavage”). Expression of the fusion protein is driven from the yeast TEF1 promoter (“P_TEF1”) and terminated with a 3× stop codon (“3× stop”). The plasmid contains a kanamycin resistance marker for E. coli selection (“Kan_R”), an origin of replication for E. coli propagation (“ColE1 ori”), a URA3 auxotrophic marker for yeast selection (“LEU2”), and a 2 um element for yeast propagation (“2 um”).

FIG. 8 is a plasmid map of a S. cerevisiae integration vector for expression of endoplasmic reticulum-targeted bovine P4HA1. The bovine (Bos taurus) P4HA1 gene with an appended 3′ HDEL peptide sequence (“Bt_P4HA1-HDEL”) was fused to the 3′ end of a Kar2 leader sequence (“Kar2”) for targeting to the endoplasmic reticulum. The fusion protein was driven from the yeast TEF1 promoter (“P_TEF1”) and terminated with a 3′ UTR from the yeast CYC1 gene (“3′UTR_CYC1”). The plasmid contains a kanamycin resistance marker for E. coli selection (“Kan_R”), an origin of replication for E. coli propagation (“ColE1 ori”), a LEU2 auxotrophic marker for yeast selection (“LEU2”), a HIS3 auxotrophic marker for yeast selection (“HIS3”), and a 2 um element for yeast propagation (“2 um”).

FIG. 9 is a plasmid map of a S. cerevisiae integration vector for expression of endoplasmic reticulum-targeted bovine P4HB. The bovine (Bos taurus) P4HB gene with an appended 3′ HDEL peptide sequence (“Bt_P4HB-HDEL”) was fused to the 3′ end of a Kar2 leader sequence (“Kar2”) for targeting to the endoplasmic reticulum. The fusion protein is driven from the yeast TEF1 promoter (“P_TEF1”) and terminated with a 3′ UTR from the yeast CYC1 gene (“3′UTR_CYC1”). The plasmid contains a kanamycin resistance marker for E. coli selection (“Kan_R”), an origin of replication for E. coli propagation (“ColE1 ori”), a URA3 auxotrophic marker for yeast selection (“URA3”), a HIS3 auxotrophic marker for yeast selection (“HIS3”), and a 2 um element for yeast propagation (“2 um”).

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

All technical terms employed in this specification are commonly used in biochemistry and molecular biology; hence, they are understood by those skilled in the field to which the present technology belongs. Those technical terms can be found, for example in: Molecular Cloning: A Laboratory Manual 3rd ed., vol. 1-3, ed. Sambrook and Russel (Cold Spring Harbor Laboratory Press, Cold Spring Harbor, N.Y., 2001); Current Protocols In Molecular Biology, ed. Ausubel et al., (Greene Publishing Associates and Wiley-Interscience, New York, 1988) (including periodic updates); Short Protocols In Molecular Biology: A Compendium Of Methods From Current Protocols In Molecular Biology 5th ed., vol. 1-2, ed. Ausubel et al., (John Wiley & Sons, Inc., 2002); Genome Analysis: A Laboratory Manual, vol. 1-2, ed. Green et al., (Cold Spring Harbor Laboratory Press, Cold Spring Harbor, N.Y., 1997).

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the technology. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, the phrase “and/or,” should be understood to mean “either or both” of the elements so conjoined, e.g., elements that are conjunctively present in some cases and disjunctively present in other cases. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein, when the term “approximately” is used in conjunction with a numerical range, it modifies that range by extending the boundaries above and below those numerical values. In general, the term “approximately” is used herein to modify a numerical value above and below the stated value by a variance of 20%, 10%, 5%, or 1%. In certain embodiments, the term “approximately” is used to modify a numerical value above and below the stated value by a variance of 10%. In certain embodiments, the term “approximately” is used to modify a numerical value above and below the stated value by a variance of 5%. In certain embodiments, the term “approximately” is used to modify a numerical value above and below the stated value by a variance of 1%.

As used herein, the term “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, the terms “biocapsule” or “filamentous fungus-yeast biocapsule” refer to a form of yeast and filamentous fungus co-immobilization. The combined inherent adhesive properties of yeast and filamentous fungus give rise to the spontaneous formation of biocapsules through hydrophobic interactions when cultured under certain conditions in a liquid medium. In some embodiments, the yeast are not genetically engineered. In some embodiments, the yeast are genetically engineered to express one or more heterologous proteins selected from one or more extracellular matrix (ECM) proteins, growth factor proteins, cell adhesion proteins, cell surface proteins, cell signaling proteins, motif peptides (synthetic or naturally-occurring), flavor proteins, or any combination thereof, wherein the heterologous protein is expressed solubly, targeted to a specific subcellular localization, or secreted. In some embodiments, the yeast are fixed or decellularized. In some embodiments, the biocapsules are used as biomaterial scaffolds. In some embodiments, the biocapsules are used as an edible biomaterial scaffold. In some embodiments, the biocapsules are used as an edible biomaterial scaffold for cell-based meat production according to the methods described herein.

As used herein, the term “embodiment” means that a particular feature, structure or characteristic is included in at least one or more manifestations, examples, or implementations of the present technology. Furthermore, the particular features, structures or characteristics may be combined in any suitable manner, as would be apparent to a person skilled in the art. Combinations of features of different embodiments are meant to be within the scope of the present technology, without the need for explicitly describing every possible permutation by example. Thus, any of the claimed embodiments can be used in any combination.

As used herein, the terms “genetic engineering” or “genetically engineered” encompass any methodology for introducing a nucleic acid or specific mutation into a host organism. For example, a yeast cell is genetically engineered when it is transformed with a polynucleotide sequence that expresses a heterologous protein. A cell is genetically engineered when a polynucleotide sequence is introduced that results in the expression of a novel and/or heterologous gene in the cell, or an increase or decrease in the level of a gene product that is naturally found in the cell through up- or down-regulation, deletion, or change in copy-number.

“Heterologous nucleic acid” refers to a nucleic acid, DNA, or RNA, which has been introduced into a cell (or the cell's ancestor), and which is not a copy of a sequence naturally found in the cell into which it is introduced. Such heterologous nucleic acid may comprise segments that are a copy of a sequence that is naturally found in the cell into which it has been introduced, or fragments thereof.

As used herein, the term “progenitor cell” refers to a cell capable of giving rise to differentiated cells in multiple lineages. Progenitor cells may include stem cells, such as embryonic stem cells, mesenchymal stem cells, adult stem cells, differentiated embryonic stem cells, differentiated adult stem cells, or induced pluripotent stem cells.

### II. General

The present technology relates to compositions and methods for the manufacture of an edible biocapsule that provides structure and a suitable growth environment for the attachment, proliferation, and morphogenesis of cultured animal cells for the production of cell-based meat products, such as steak-like cultivated meat products. The biocapsule comprises an edible base substrate and yeast cells (such as, e.g., baker's yeast (Saccharomyces cerevisiae)). In some embodiments, the yeast of the biocapsule is not modified or genetically engineered. In some embodiments, the yeast of the biocapsule is genetically engineered to express one or more heterologous proteins (such as, e.g., one or more extracellular matrix (ECM) proteins, growth factor proteins, cell adhesion proteins, cell surface proteins, cell signaling proteins, motif peptides (synthetic or naturally-occurring), flavor proteins, or any combination thereof). The base substrate comprises the mycelium of an edible filamentous fungus and can be grown into any desired shape and size, and serves as the main structural element of the final cultivated meat product. The base substrate is functionalized with yeast, which can optionally be genetically engineered. The base substrate can be functionalized with recombinant proteins expressed by the genetically-engineered baker's yeast, for example, which is composed largely of an edible polysaccharide. The engineered baker's yeast can be decellularized or fixed, such that the yeast are rendered inert while the heterologous proteins, which may be expressed solubly, targeted to a specific subcellular localization, or secreted, remain structurally intact.

### III. Scaffolds for Animal Tissue Growth

Animal tissues are organized arrangements of various cell types grown into a three-dimensional structure. For individual cells to grow into an organized tissue, the cells may adhere to some type of supportive scaffold that promotes expansive growth in three dimensions. Scaffolding biomaterials may possess biocompatible surfaces to mimic the structure of the tissue in three dimensions, and exhibit interconnected porosity to support cell/tissue penetration. Scaffolds made from natural sources are advantageous because of their innate ability to promote biological recognition, and support cell adhesion and function.

Naturally derived scaffold biomaterials are generally divided into two groups: (1) extracellular matrix (ECM) protein-based biomaterials such as collagen, silk fibroin, gelatin, fibronectin, keratin, fibrin, and eggshell membrane; and (2) polysaccharide-based biomaterials such as hyaluronan, cellulose, glucose, alginate, chondroitin, and chitin and its derivative, chitosan. While both groups of scaffold proteins are important for tissue growth, most of the developments have only been made on the polysaccharide-based biomaterial front. Although progress has been made in the development of tunable scaffolds made of food-safe, plant-derived polymers like gellan gum, alginate, pectin, and modified cellulose, ECM proteins are, however, currently still mostly harvested from animal sources, which is inefficient and suffers from batch-to-batch variance. In addition, current methods in the art to produce scaffolds that combine both ECM proteins and polysaccharide-based biomaterials use functionalization techniques that require highly purified ECM proteins, which is extremely costly and inefficient.

As described herein, the present technology relates to the use a fungi-based approach for the production of robust, customizable scaffolds that have the polysaccharide-based structural elements and/or proteins, such as ECM proteins, that may improve the growth and proliferation of any given animal cell type into any desired tissue.

### IV. Fungi

Molds and yeasts are fungi widely distributed in nature. A fungus is any member of the group of eukaryotic organisms that includes microorganisms such as yeasts and molds, as well as the more familiar mushrooms. Molds or filamentous fungi are presented as long filaments of cells (hypha) that form a mycelium. Hypha are long, branching filamentous structures of a fungus. Filamentous fungi are comprised of cross-linked networks of hyphae, which expand via polarized tip extension and branch formation (increasing the number of growing tips), which is equivalent to cell division in animals and plants. In most fungi, hyphae are the main mode of vegetative growth, and are collectively called a “mycelium.”

It should be appreciated that the mycelium of the present technology may be grown by any method known to those having ordinary skill in the art.

In some embodiments, the mycelial scaffold may be generated by using a perfusion bioreactor system for cell-based meat technologies. The perfusion bioreactor system may include, for example, suspension reactor units for beef myocyte propagation, dialysis, oxygenation, pumps for media cycling between reactor units and media feeding, and scaffold bioreactor units for producing agglomerated cell masses with or without mechanical actuation of the agglomerated cellular mass.

In some embodiments, the filamentous fungus comprises one or more strains belonging to a division selected from the group consisting of Ascomycota, Basidiomycota, and Zygomycota (Mucoromycota and Zoopagomycota). In some embodiments, the filamentous fungus strain is from the Ascomycota division and comprises one or more strains belonging to a genus selected from the group consisting of Aspergillus, Penicillium, and Neurospora. In some embodiments, the filamentous fungus is from the Aspergillus genus and comprises one or more strains of Aspergillus oryzae. In some embodiments, the filamentous fungus is from the Neurospora genus and comprises one or more strains of Neurospora crassa. In some embodiments, the filamentous fungus is a strain of Rhizopus oryzae or Rhizopus oligosporus from the Mucoromycota division.

Molds, such as Penicillium and Aspergillus, are aerobic and, due to their chemical activities, are very important in the production of antibiotics, cheeses, etc. Penicillium is a genus of ascomycetes fungi that is of importance in the natural environment, in food spoilage, and in food and drug production. Aspergillus is the genus comprising a group of filamentous fungi or common molds, most of which occur in an asexual state, and reproduce by producing conidia (asexual spores or conidiophores) that can spread into many different environments, germinate, and grow.

A yeast is a eukaryotic, single-celled microorganism classified as a member of the fungus kingdom. Saccharomyces cerevisiae (commonly known as brewer's yeast) is a species of anaerobic yeast that is instrumental in winemaking, baking, and brewing. Some Saccharomyces cerevisiae can form a veil or flower on the surface of the wine once the alcoholic fermentation is finished and the fermentable sugars are depleted, which is a form of spontaneous immobilization.

Brewer's yeast can be easily cultured as a liquid at room temperature, grows rapidly (˜2 hours doubling time), and its genome can be easily manipulated using well-established molecular biology toolsets. As a eukaryotic organism, brewer's yeast is equipped with the cellular machinery capable of efficiently producing mammalian proteins. Brewer's yeast is edible, and best known for its roles in baking and brewing, in alcohol production, and is even consumed as a nutritional supplement due to its numerous health benefits.

Aspergillus oryzae (commonly known as koji) is safe for human consumption, as it is traditionally used to make sake, soy sauce, and miso, and has a natural umami flavor which resembles that of meat. As a standalone material or in conjunction with budding yeast, the fibrous material from koji's fungal mycelium can form a vegetative structure of fungi composed of branching, thread-like hyphae. Koji can be grown to form solid structures of any size.

Additionally, the density and material properties of koji can be tuned during production, and if necessary, this filamentous fungus is amenable to genetic engineering. Accordingly, in some embodiments of the present technology, koji constitutes the main chitin structural element for the scaffold, providing the architectural skeleton for the animal tissue. The addition of engineered brewer's yeast to the koji scaffolds will add customizable properties that can be used to improve the adherence and proliferation of various animal cells into a functional tissue.

### V. Chitin

Chitin is the main component of the cell wall of budding yeast and filamentous fungi. Chitin is a naturally abundant polysaccharide that is the product of de-acetylated chitosan, another naturally abundant polymer. Chitin/chitosan is the main component of the shells of crustaceans such as shrimp, crab, and lobster, and is also the principle component of the cell wall of various fungi including Aspergillus oryzae (commonly known as Koji), Saccharomyces cerevisiae (commonly named brewer's, baker's or budding yeast). Chitin can be readily manipulated into three-dimensional scaffolds as a substrate for animal tissue culturing. In addition to its tractable chemical properties, animal-free nature, and abundance, chitin/chitosan also contains antimicrobial properties and nutritional benefits. As described herein, in some embodiments, chitin/chitosan sourced from both baker/brewer's yeast and Koji is used as the main structural component of the customized scaffolds engineered specifically for each animal tissue.

As a standalone material or in conjunction with budding yeast, the fibrous material from koji's fungal mycelium can form a vegetative structure of fungi composed of branching, thread-like hyphae. Koji can be grown to form solid structures of any size using the procedures known in the art. Additionally, its density and material properties can be tuned during production, and if necessary, this filamentous fungus is amenable to genetic engineering. Accordingly, as described herein, in some embodiments, koji will constitute the main chitin structural element for the biomaterial scaffold, providing the architectural skeleton for the animal tissue (e.g., the shape of a steak). The addition of engineered brewer's yeast to the koji scaffolds adds customizable properties that can be used to improve the adherence and proliferation of various chordate and non-chordate cells into a functional tissue.

### VI. Yeast and Genetically Engineered Yeast

As described herein, the biocapsule compositions and methods of the present technology comprise yeast cells. Those yeast calls can optionally be genetically engineered to express one or more heterologous proteins for animal cell adhesion and subsequent cell activity. In some embodiments, the yeast comprises one or more strains selected from the group consisting of Saccharomyces cerevisiae, Saccharomyces bayanous, Saccharomyces capensis, Schizosaccharomyces pombe, and Pichia pastoris. In some embodiments, the yeast comprises one or more strains of Saccharomyces cerevisiae (baker's yeast; brewer's yeast).

In some embodiments, the one or more heterologous proteins are selected from one or more extracellular matrix (ECM) proteins, growth factor proteins, cell adhesion proteins, cell surface proteins, cell signaling proteins, motif peptides (synthetic or naturally-occurring), flavor proteins, or any combination thereof. In some embodiments, the heterologous proteins are selected from one or more chordate or non-chordate sea creature ECM proteins, growth factor proteins, cell adhesion proteins, cell surface proteins, cell signaling proteins, motif peptides (synthetic or naturally-occurring), flavor proteins, or any combination thereof. In some embodiments, the heterologous chordate proteins are mammalian ECM proteins, growth factor proteins, adhesion molecule proteins, cell surface proteins, cell signaling proteins, motif peptides (synthetic or naturally-occurring), flavor proteins, or any combination thereof. In some embodiments, the heterologous protein comprises one or more ECM proteins selected from the group consisting of: collagen, laminin, fibronectin, and RGD motif peptides. Flavor proteins include those proteins that impact the flavor of a composition, for example through the binding and/or absorption of flavor imparting compounds. The scope of appropriate flavor proteins, and how they may impact a compositions flavor, will be appreciated by one with skill in the art. See Zhang, Jian, Kang, Dachend, Zhang, Wangang, and Lorenzoe, Jose. Recent advantage of interactions of protein-flavor in foods: Perspective of theoretical models, protein properties and extrinsic factors. Trends in Food Science and Technology. 2021 May;111:405-425. In some embodiments, the heterologous protein is expressed solubly, targeted to a specific subcellular localization, or secreted.

In some embodiments, the yeast cells can be employed either living or dead, intact, permeabilized, or even emptied of all their original cytoplasmic contents (decellularized). In some embodiments, the yeast cells of the present technology are fixed. The yeast cells may be fixed or decellularized by any suitable method known in the art, including but not limited to, fixation or decellularization by thermal shock, treatment with detergent, dehydrating agent such as alcohol, osmotic shock, lyophilization, physical (mechanical) lysing, electrical disruption, enzymatic digestion, or any combination thereof.

### VII. Encapsulation

Encapsulation involves the incorporation of food ingredients, enzymes, cells or other materials in small capsules. Applications for this technique have increased in the food industry since the encapsulated materials can be protected from moisture, heat or other extreme conditions, thus enhancing their stability and maintaining viability. Various techniques are employed to form the biocapsules, including spray drying, spray chilling or spray cooling, extrusion coating, fluidized bed coating, liposome entrapment, coacervation, inclusion complexation, centrifugal extrusion and rotational suspension separation. A wide variety of food is encapsulated: flavoring agents, acids bases, artificial sweeteners, colorants, preservatives, leavening agents, antioxidants, agents with undesirable flavors, odors and nutrients, among others. Micro-encapsulations, or the process in which tiny particles or droplets are surrounded by a coating to give small capsules, have also been performed, especially for the immobilization of enzymes because a large number of enzymes can be trapped with synthetic polymers (such as cellulose acetate thalate) without denaturing or undergoing chemical modifications.

The combination of the fixed yeasts containing the heterologous proteins and the filamentous fungus-based (e.g., koji-based) scaffold takes advantage of the inherent adherent properties of both of these fungi. Brewer's yeast associates with hyphae of filamentous fungi naturally through strong hydrophobic interactions.

The inherent adherent properties of the Eumycetes (a division of fungi that includes all true fungi (ascomycetes and basidiomycetes) as distinguished from the slime molds) used in the process allows for the functionalization of the koji-based substrate with the yeast cells. Baker's yeast associates with hyphae of filamentous fungi naturally through strong hydrophobic interactions. Moreover, protocols to induce co-immobilization of a filamentous fungus (e.g., koji) and brewer's yeast without adding chemical cross-linkers or external supports have been previously described. Techniques have been demonstrated to produce large spheres (˜2 cm diameter) containing both microorganisms, i.e. “filamentous fungus-yeast biocapsules,” that are extremely stable under a number of harsh conditions and resemble established scaffolds.

The density and porosity of these biocapsules can be modulated by taking advantage of an inherent property of glucan, a main component of the budding yeast cell wall. A glucan is a polysaccharide derived from D-glucose, linked by glycosidic bonds. Glucans can polymerize and form a gel at neutral pH, thus adding another layer of tunability to the system. A biomaterial scaffold composed of an edible and structurally robust koji-based matrix and brewer's yeast, optionally engineered to express heterologous proteins such as extracellular matrix (ECM) proteins, growth factor proteins, cell adhesion proteins, cell surface proteins, cell signaling proteins, motif peptides (synthetic or naturally-occurring), flavor proteins or any combination thereof, as described herein, provides the three-dimensional architecture and cell-proliferation cues for the generation of muscle/fat-based animal tissues.

### VIII. Plurality of One or More Cell Types, Seeding, and Culturing the Cells

Numerous cell types or populations of cells may be cultured with the filamentous fungus-yeast biocapsules of the present technology, so as to form a three-dimensional edible cell-based meat product. In some embodiments, the one or more cell types are selected from the group consisting of: myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, mesenchymal stem cells, embryonic stem cells, and any combination thereof.

In some embodiments, the plurality of cell types are progenitor cells. In some embodiments, the progenitor cells are cultured in monoculture. In some embodiments, the progenitor cells are differentiated in a monoculture. In some embodiments, the progenitor cells are differentiated in a monoculture and then seeded and incubated on the three-dimensional filamentous fungus-yeast biocapsules of the present technology. In some embodiments, the progenitor cells are seeded and incubated on the three-dimensional filamentous fungus-yeast biocapsules of the present technology and differentiated in situ. Methods of culturing and differentiating progenitor cells to differentiated cells would be readily apparent to those of ordinary skill in the art.

In some embodiments, the plurality of cell types are obtained from a live animal and cultured as a primary cell line. In some embodiments, the cells are obtained by biopsy and cultured ex vivo. In some embodiments, the cells are obtained from commercial cell lines. In some embodiments, the cells are immortalized or reprogrammed primary cell lines.

In some embodiments, the plurality of cell types are derived from chordates. In some embodiments, the plurality of cell types are derived from non-human chordates selected from mammals, birds, fish, reptiles, amphibians. In some embodiments, the plurality of cell types are derived from non-human mammals. In some embodiments, the plurality of cell types are derived from livestock, which includes, for example, domestic, semi-domestic, and captive wild animals, including, but not limited to, cattle, elk, reindeer, bison, horses, deer, sheep, goats, swine, poultry, llamas, alpaca, and live fish. In some embodiments, the plurality of cell types are derived from non-chordate sea creatures. In some embodiments, the plurality of cell types are derived from non-chordate sea creatures selected from, but not limited to, crustaceans (e.g., lobster, crab, shrimp, clams, oysters, mussels), sea urchins, squids, and octopus. In some embodiments, the plurality of one or more cells is derived from the same chordate or non-chordate as the heterologous protein that can be expressed by the yeast cells of the biocapsules of the present technology. In some embodiments the plurality of one or more cells and the heterologous protein that can be expressed by the yeast cells of the biocapsules of the present technology are derived from mammals. In some embodiments, the plurality of one or more cells and the heterologous protein that can be expressed by the yeast cells of the biocapsules of the present technology are either a homogenous or heterogeneous combination of the chordates or non-chordates described herein. In some embodiments, the plurality of one or more cells and the heterologous protein that can be expressed by the yeast cells of the biocapsules of the present technology are either a homogenous or heterogeneous combination of porcine, bovine, or poultry cell types.

Those of skill in the art will readily recognize that each of the plurality of cell types used in the compositions and methods of the present technology have preferred media for growing and maintaining the cells and also have a preferred range of cell density.

In some embodiments, the biocapsules of the present technology are seeded with a plurality of cell types in a range of about 1×10³ cells to about 1×10⁶ cells on a scaffold ranging from about 1 cm² to 60 cm² total surface area in about 1 mL to about 10 mL media suitable for growing and maintaining the cells. In some embodiments, the biocapsules are seeded with about 1×10⁵ cells on about 9 cm² total surface area of scaffold in about 3 mL media.

In some embodiments, “coverage %” refers to the area or volume of a biocapsule scaffold that is in contact with the plurality of one or more cell types. In another embodiment, coverage % refers to the area or volume of a biocapsule scaffold that is occupied by the plurality of one or more cell types. As used herein, cells in contact with the biocapsule scaffold are on, within, or a combination thereof.

In some embodiments, coverage % of the plurality of cell types is at least 10%, at least 20%, at least 30%, at least 40%, at least 50%, at least 60%, at least 70%, at least 80%, at least 90%, or at least 99%. In some embodiments, coverage % of the plurality of cells is 5-20%, 15-30%, 25-40%, 35-50%, 45-60%, 55-70%, 65-80%, 75-90%, 85-100%, or any range therebetween.

The skilled artisan will appreciate that the seeding and/or the culturing of cells is performed in the presence of a cell culture medium. In some embodiments, the cell culture medium comprises growth factors, cytokines, bioactive agents, nutrients, amino acids, antibiotic compounds, anti-inflammatory compounds, or any combination thereof. Suitable medium and compounds suitable for viability and growth of the cells are known to one skilled in the art.

Growth factors or fragments thereof that can be used in the methods and compositions of the present technology include, but are not limited to, platelet-derived growth factors (PDGF) and insulin-like growth factor (IGF-1). PDGF and IGF-1 are known to stimulate mitogenic, chemotactic and proliferate (differentiate) cellular responses. The growth factor can be, but is not limited to, one or more of the following: PDGF, e.g., PDGF AA, PDGF BB; IGF, e.g., IGF-I, IGF-II; fibroblast growth factors (FGF), e.g., acidic FGF, basic FGF, β-endothelial cell growth factor, FGF 4, FGF 5, FGF 6, FGF 7, FGF 8, and FGF 9; transforming growth factors (TGF), e.g., TGF-P1, TGF β, TGF-p2, TGF-p3, TGF-p5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, betacellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, ciliary neurotrophic factor, and neuregulin (NRG1), or fragments thereof.

In some embodiments, the plurality of cells types are incubated with the edible biocapsule scaffold. The scaffold may be in a particular shape or form so as to influence or delimit a three-dimensional shape or form assumed by a population of proliferating cells. In some embodiments, the biocapsule scaffold mimics the three-dimensional shape of a meat product.

### IX. Method for Generating a Filamentous Fungus-Yeast Biocapsule

As described herein, the technology of the present disclosure relates to methods for generating a filamentous fungus-yeast biocapsule. In some embodiments, the filamentous fungus-yeast biocapsule serves as an edible biomaterial scaffold that provides three-dimensional structure and a suitable growth environment for the attachment, proliferation, and morphogenesis of cultured animal cells for the production of cultivated meat products.

The method for generating the biocapsules of the present technology comprises inducing a spontaneous co-immobilization between a filamentous fungus and a plurality of yeast cells, in the absence of chemical binding compounds and external supports, to artificially create the appropriate conditions to favor corresponding symbiosis. The combined inherent adhesive properties of yeast and filamentous fungus give rise to the spontaneous formation of biocapsules through hydrophobic interactions when cultured under certain conditions in a liquid medium. In some embodiments, the yeast are genetically engineered to express one or more heterologous proteins selected from one or more extracellular matrix (ECM) proteins, growth factor proteins, cell adhesion proteins, cell surface proteins, cell signaling proteins, motif peptides (synthetic or naturally occurring), flavor proteins, or any combination thereof, wherein the heterologous protein is expressed solubly, targeted to a specific subcellular localization, or secreted. In some embodiments, the yeast are fixed. In some embodiments, the yeast are decellularized.

In some embodiments, the filamentous fungus comprises one or more strains belonging to a division selected from the group consisting of Ascomycota, Basidiomycota, and Zygomycota (Mucoromycota and Zoopagomycota). In some embodiments, the filamentous fungus strain is from the Ascomycota division and comprises one or more strains belonging to a genus selected from the group consisting of Aspergillus, Penicillium, and Neurospora. In some embodiments, the filamentous fungus is from the Aspergillus genus and comprises one or more strains of Aspergillus oryzae (koji). In some embodiments, the filamentous fungus is from the Neurospora genus and comprises one or more strains of Neurospora crassa. In some embodiments, the filamentous fungus is from the Rhizopus genus and comprises one or more strains of Rhizopus oryzae or Rhizopus oligosporus. In some embodiments, the yeast comprises one or more strains selected from the group consisting of Saccharomyces cerevisiae, Saccharomyces bayanous, Saccharomyces capensis, Schizosaccharomyces pombe, and Pichia pastoris. In some embodiments, and a species of the yeast is Saccharomyces cerevisiae, Saccharomyces bayanous, or Saccharomyces capensis, the yeast comprises one or more strains of Saccharomyces cerevisiae. It should be appreciated that the specific examples of filamentous fungi and yeast are not limited to those explicitly listed herein and that other examples are contemplated.

The method comprises creating conditions to induce co-immobilization between the filamentous fungus and yeast cells. The conditions to induce the co-immobilization between the filamentous fungus and yeast cells comprise the use of a first culture medium and a second culture medium. The first culture medium comprises a carbon source usable by the filamentous fungus and not usable by the yeast, where the carbon source is selected from gluconic acid, starch, cellulose, inulin, or other similar colloidal molecules. The second culture medium comprises a preferred carbon source for the yeast that is also usable by the filamentous fungus. Agitation can be carried out with an orbital stirrer, in a thermostat with a stirring-aeration system, sparging, or with any other means that is suitable for agitation.

External factors or conditions of the method for generating the biocapsules of the present technology may include: an incubation temperature between approximately 20° C. to approximately 37° C., an agitation speed between approximately 0 rpm and approximately 270 rpm, and a time period of 24 hours or more for cultivation. It should be appreciated that these external factors are provided for explanatory purposes only.

The biocapsules generated by the methods of the present technology may, in some embodiments, be used in the manufacture of cell-based meat products. Accordingly, in some embodiments, the methods may further comprise seeding the filamentous fungus-yeast biocapsule with a plurality of one or more cell types selected from the group consisting of: myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, mesenchymal stem cells, embryonic stem cells, and any combination thereof, to form a seeded biocapsule. The methods may further comprise culturing the seeded biocapsule under conditions effective to induce differentiation of the plurality of one or more cell types to produce an edible cell-based meat product.

In some embodiments, the method may also comprise engineering the yeast cells to express at least one heterologous protein, protein fragment, and/or protein motif to impart one or more characteristics to the filamentous fungus. In some embodiments, the heterologous protein is expressed solubly, targeted to a specific subcellular localization, or secreted. In some embodiments, the heterologous protein comprises one or more heterologous proteins selected from extracellular matrix (ECM) proteins, growth factor proteins, cell adhesion proteins, cell surface proteins, cell signaling proteins, motif peptides (synthetic or naturally occurring), flavor proteins, or any combination thereof. In some embodiments, the ECM proteins are selected from one or more of collagen, laminin, fibronectin, and RGD motif peptides. In some embodiments, the heterologous proteins are of non-human chordate origin. In some embodiments the heterologous proteins are of non-human mammalian origin. In some embodiments the heterologous proteins are of porcine origin. In some embodiments the heterologous proteins are of bovine origin. In some embodiments the heterologous proteins are of poultry origin. In some embodiments, the heterologous proteins are of non-chordate sea creature origin. A non-exhaustive list of examples include: adding collagen to impart a jelly structure and adding fibronectin to impart cell-adhesion.

As such, the desired one or more characteristics of the edible biocapsule scaffolds described herein are engineered into the biocapsule scaffolds and are inherent to the scaffolds, whereas others in the technical field modify their scaffolds through chemistry by induction, deposition, or infusion of exogenous materials and biomaterials, such as hormones, minerals, or agarose/gelatins to impart the desired characteristics onto the scaffolds. Furthermore, the scaffolds of the present technology are tunable and can be designed to, for example, specifically allow co-culturing of muscle and fat cells, and express growth factors that can improve cell proliferation and differentiation, as well to potentially reduce manufacturing costs.

In some embodiments, the methods comprise shaping the scaffold into any desired shape. In some embodiments, the shape of the scaffold mimics the shape of a three-dimensional meat product.

In some embodiments, the methods comprise introducing an enzyme (e.g., zymolyase, lyticase, and/or glucalase, among others) to the filamentous fungus to modify a parameter of the filamentous fungus, such as the density, porosity, and/or texture of the filamentous fungus.

In some embodiments, the methods comprise producing the filamentous fungus-yeast biocapsule. In some embodiments, the filamentous fungus-yeast biocapsule is a hollow capsule of a shape (e.g., a shape that mimics a shape of a meat product), whose walls limit an interior space partially occupied by free yeast cells and others associated with hyphae, forming clusters, leaving the culture medium transparent. In some embodiments, the scaffold will have the shape of the final meat product, e.g., slices of meat, and as such, the shape will be a laminal shape with micro-pores or striations. It should be appreciated that the shape of the biocapsule is not limited and any shape is contemplated. The size of the biocapsules may depend on several external factors, such as agitation speed, agitation time, and incubation temperature. The diameter of the biocapsules can vary from between a few millimeters and several centimeters. The three-dimensional structure of a biocapsule is not limited to a spherical shape and can adopt the shape of the vessel in which it is cultured.

In some embodiments, the biocapsules described herein are edible biomaterial scaffolds that provide structure and a suitable growth environment for the attachment, proliferation, and morphogenesis of cultured animal cells for the production of steak-like cultivated meat products. In some embodiments, the scaffold comprises an edible base substrate (filamentous fungus) that is functionalized with yeast cells. In some embodiments, the base substrate is functionalized with heterologous proteins expressed by genetically-engineered yeast cells (e.g., baker's yeast). In some embodiments, the base substrate comprises the mycelium of an edible filamentous fungus and can be grown into any desired shape and size, and serves as the main structural element of the final cultivated meat product. In some embodiments, the base substrate is functionalized with heterologous proteins expressed by genetically-engineered yeast (e.g., baker's yeast), which is composed largely of an edible polysaccharide. In some embodiments, the baker's yeast will be decellularized or fixed.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: Making Biocapsules

Yeast is grown in YP+3% glycerol. Aspergillus is grown on plates, where spores are harvested with BFM. BFM (0.67% YNB+0.5% gluconic acid) is inoculated with varying ratios of yeast and spores. Growth occurs at 30° C. with shaking until biocapsules reach a desired size. Next, the biocapsules are grown in YP+18% glucose. The biocapsules are then harvested and washed in water.

### Example 2: Ethanol Fixation Protocol

Scaffolds (biocapsules) are washed with 2×50 mL water for 10 minutes and are then incubated with 50 mL of 40% ethanol for 60-180 minutes. Then, the scaffolds are transferred to sterile tubes, which are washed in sterile water and stored in sterile PBS.

### Example 3: C2C12 Seeding, Growing, Differentiating on Scaffolds

This example utilizes C2C12, which is an immortalized mouse myoblast cell line. Developed for in vitro studies of myoblasts isolated from the complex interactions of in vivo conditions, C2C12 cells are useful in biomedical research, as these cells are capable of rapid proliferation under high serum conditions and differentiation into myotube fibers under low serum conditions.

C2C12 cells are grown to 80-90% confluence in growth medium (DMEM+10% FBS). The cells are trypsinized and washed in growth medium. Next, seeding at 40-50% confluence occurs directly on to the scaffold, such as a biocapsule scaffold generated in Example 1. Growth occurs for 7 days with DMEM+FBS, where the media is replenished every 2 days. Optionally, some sample is sacrificed and examined by phalloidin/nuclear stain to determine cell proliferation. On day 7, switching to differentiation media (DMEM+2% horse serum) occurs. Then, growth occurs for 5-7 days, where media is replenished every 4 days. Optionally, some sample is sacrificed and examined by phalloidin/nuclear stain to determine differentiation/myotube formation.

### Example 4: Biocapsule Growth Using Solid Substrate

Aspergillus is grown on plates and spores are harvested in BFM. Yeast is grown in YP+3% glycerol. Varying ratios of Aspergillus spores and yeast are suspended in water and spread onto BFM agar. Plates are incubated at 28° C. for 72 hours. BFM is aspirated off and is replaced with YPD18 (18% glucose). Incubation occurs 28° C. for 24 hours. Biocapsule on the surface of agar is harvested and processed.

### Example 5: Use of a Filamentous Fungus-Yeast Biocapsule for the Production of a Cell-Based Meat Product

This example will demonstrate that the filamentous fungus-yeast biocapsules of the present technology are useful in methods for producing cell-based meat products.

Briefly, genetically engineered Saccharomyces cerevisiae (baker's yeast) cells are added to a culture of Aspergillus oryzae (koji) comprising mycelia to form a filamentous fungus-yeast preparation. The yeast cells are genetically engineered to express one or more heterologous proteins. The heterologous proteins may be expressed solubly, targeted to a specific subcellular localization, or secreted. The heterologous protein may comprise any one or more chordate ECM proteins (e.g., collagen, laminin, fibronectin, RGD motif peptides), growth factor proteins, cell adhesion proteins, cell signaling proteins, peptide motifs (synthetic or naturally-occurring), or cell surface proteins. The preparation is then cultured under conditions (as described herein) that are sufficient to induce spontaneous co-immobilization of the Aspergillus oryzae and Saccharomyces cerevisiae, thereby forming a filamentous fungus-yeast biocapsule. The filamentous fungus-yeast biocapsules are then processed by fixation, decellularization, or other means to inhibit further growth while keeping endogenous protein structures intact.

The inert filamentous fungus-yeast biocapsules are then seeded with a plurality of one or more cell types selected from myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, mesenchymal stem cells, embryonic stem cells, or any combination thereof, to form a seeded biocapsule. The seeded biocapsule is then cultured under conditions that are effective to induce the growth and/or differentiation of the plurality of the one or more cell types to produce an edible cell-based meat product.

Accordingly, these results will demonstrate that the filamentous fungus-yeast biocapsules of the present technology are effective in methods for producing cell-based meat products.

### Example 6: Co-Immobilization of Baker's Yeast and Koji Mycelium

Introduction. This experiment demonstrates the generation of a biocapsule/scaffold for use in production of a cell-based meat product. In particular, this examples demonstrates the co-immobilization of Saccharomyces cerevisiae (also known as baker's yeast) and a filamentous fungus, Aspergillus oryzae (also known as koji).

Methods. Briefly, the spontaneous co-immobilization of a filamentous fungus and a yeast occurs when both the fungi and yeast are grown in a carbon source that can only be utilized by the filamentous fungus. As a result, the yeast become strongly associated with the hyphae of the filamentous fungi through strong hydrophobic interactions. The resulting biocapsules can be grown into a variety of shapes and sizes, most commonly spheres that can become 1-2 centimeters large. To determine the extent of co-immobilization, the co-immobilized koji/yeast symbiotic growth was washed extensively in different solvents of varying ionic strength and polarity with excessive agitation. Liquid media with a carbon source only utilized by A. oryzae (0.67% yeast nitrogen base with amino acids, 0.5% gluconic acid) was inoculated with an equimolar ratio of A. oryzae spores and S. cerevisiae and grown overnight at 30° C. with vigorous shaking to germinate the filamentous fungus. The next day, an equal volume of 2× rich glucose-containing media (2% yeast extract, 4% peptone, 32% glucose) was added to the existing co-culture and incubated at 30° C. with vigorous shaking for 1-2 days. Biocapsules composed of both fungi were then harvested and washed in a gross excess of water with vortexing. Biocapsules were then incubated with 40% ethanol, 100 mM NaCl, or 300 mM NaCl for 90-120 minutes with agitation. The biocapsules were then washed extensively in water and examined under the microscope for the presence of S. cerevisiae on the Aspergillus hyphae.

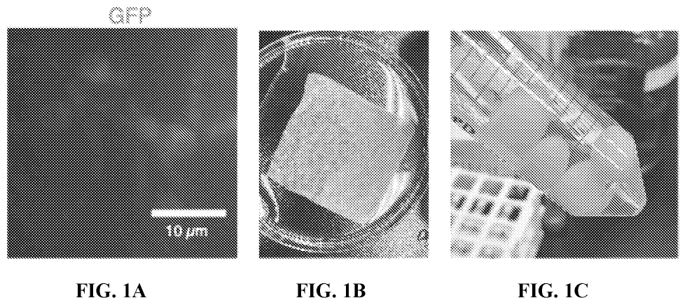

Results. This experiment resulted in the co-immobilization of A. oryzae (koji) and S. cerevisiae (baker's yeast). As shown in FIG. 1A, although some of the yeast dissociated from the filamentous fungus after the extensive washing, many remained adsorbed/adhered to the koji mycelium when examined by fluorescence microscopy. As shown in FIGS. 1B and 1C, the scaffold can be grown into a variety of shapes and densities, from flat sheets (FIG. 1B) to spheres (FIG. 1C).

Accordingly, these results demonstrate that the methods of the present technology are useful for generating biocapsules that may be used for the production of a cell-based meat product, wherein the biocapsule comprises a filamentous fungus and a plurality of yeast cells.

### Example 7: Generation of Structured Cultivated Meat Product Using Non-Engineered Yeast

Scaffold generation—for flat sheets. 5 mL liquid YPD16 (1% yeast extract, 2% peptone, 16% glucose) was inoculated with baker's yeast strain and grown at 30° C. with shaking for 16 hours. A. oryzae spores were grown BFM plates (0.67% yeast nitrogen base, 0.5% gluconic acid, 2% agar) for 2-3 days. A. oryzae spores were harvested from plates with sterile water. An equimolar mixture of yeast and A. oryzae spores in sterile water was plated onto YPD agar plate ((1% yeast extract, 2% peptone, 2% glucose, 2% agar) with a thin film of cellophane overlaid on top of the agar. The plate was incubated at 30° C. for 1-2 days until a fuzzy layer was visible on top of the overlaid cellophane. The mycelium-yeast symbiotic growth structure was then peeled off the cellophane layer and transferred to a sterile flask containing 50 mL YPD16 and incubated at 30° C. without shaking for 16-24 hours. The flask was then transferred to a shaker and grown an additional 16-24 hours at 30° C. with gentle shaking. The mycelium-yeast symbiotic growth was then harvested by washing with an excess of sterile water and incubated with 40% ethanol for 90 minutes. The scaffolds were then stored in PBS. Flat scaffolds were cut into desired shapes using a sterile scalpel.

Scaffold generation—for spheres. 5 mL liquid YPD16 (1% yeast extract, 2% peptone, 16% glucose) was inoculated with baker's yeast and grown at 30° C. with shaking for 16 hours. A. oryzae spores were grown on BFM plates (0.67% yeast nitrogen base, 0.5% gluconic acid, 2% agar) for 2-3 days. A. oryzae spores were harvested from plates with sterile water. 5 mL YPD16 was inoculated with an equimolar mixture of yeast and A. oryzae spores in sterile water and grown with vigorous shaking at 30° C. for 16-24 hours. The spherical mycelial-yeast symbiotic growth structures were then harvested by washing with an excess of sterile water and incubated with 40% ethanol for 90 minutes. The spherical scaffolds were then stored in PBS.

Cell seeding and growth. Flat scaffolds were cut into 35 mm diameter circles and transferred to the wells of a 6-well tissue culture plate. Scaffolds were washed with 3 mL growth media (GM: DMEM high glucose, no pyruvate+10% FBS+1% pen/strep). A 3 mL suspension of quail myoblast (QM7) cells at 4×10⁵ cells/mL in GM were then added dropwise to the scaffolds in the tissue culture well. Cells on scaffolds were incubated in a humidified incubator at 37° C. with 5% CO2. 3 mL fresh GM was replaced every other day for 2-3 weeks. A cell-laden scaffold was sacrificed at day two to assay for cell adhesion via fluorescence microscopy.

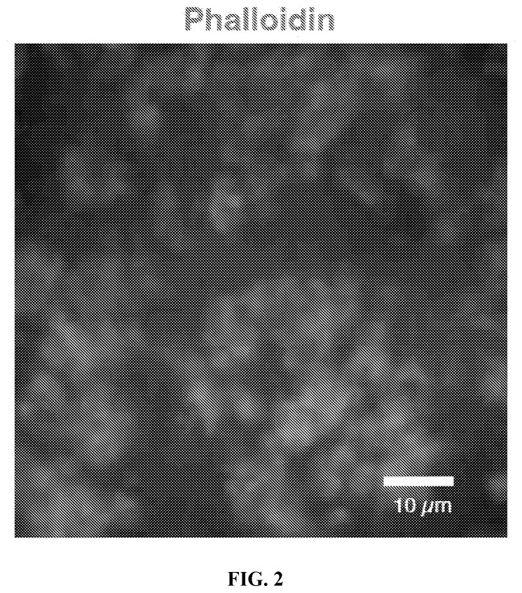

Fluorescence confocal microscopy. A cell-laden scaffold was transferred to a fresh 35 mm petri dish. Cells were fixed with 1 mL 4% paraformaldehyde for 20 minutes at room temperature. Cell-laden scaffold was then incubated in PBS+0.2% triton X-100 for 15 minutes at room temperature. The cell-laden scaffolds were then incubated in a primary antibody solution of 543-phalloidin and DAPI in PBS for 30-60 minutes at room temperature. The cell-laden scaffolds were washed twice in PBS with a 15 minute incubation at room temperature. Stained cell-laden scaffolds were then transferred to a microscope slide, sandwiched with a coverglass, then imaged by fluorescence confocal microscopy to confirm animal cell adhesion to the scaffold (FIG. 2).

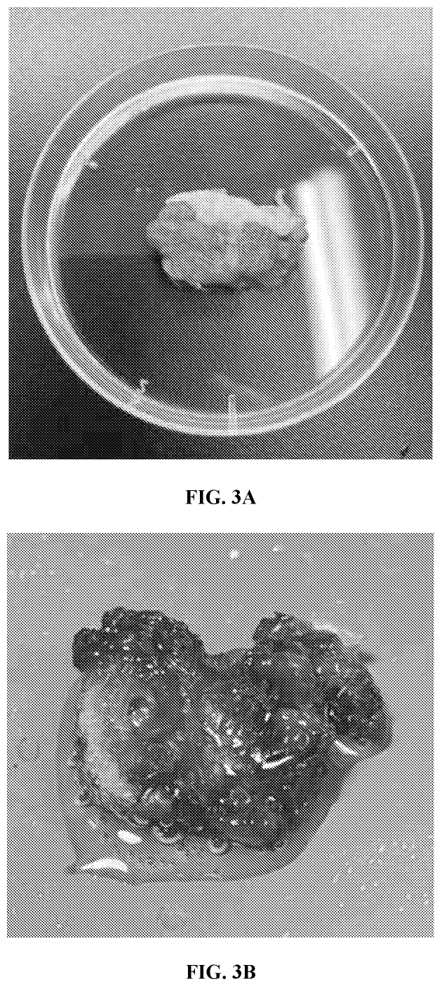

Assembly of cultivated meat product. Cell-laden scaffolds were washed extensively with sterile PBS at harvest. Some scaffolds were briefly dyed with a 1% carmine solution to impart red coloring, then washed with PBS to wash away free dye. The red and white cell-laden scaffolds were dusted with a casein+microbial transglutaminase powder mixture (MooGloo RM). The dusted scaffolds were then layered on top of one another into a three-dimensional shape. The mass was then incubated at 37° C. for 16 hours to allow the transglutaminase to “glue” the pieces together (FIG. 3A). The cultivated meat product was cooked in a pan with avocado oil (FIG. 3B).

Results. This experiment resulted in the generation of a structured cultivated meat product as shown in FIGS. 3A and 3B.

Accordingly, these results demonstrate that the methods of the present technology are useful for generating cell-based meat products comprising an edible biomaterial scaffold comprising a filamentous fungus and a plurality of yeast cells, and a plurality of one or more cell types, such as myoblasts.

### Example 8: Generation of Structured Cultivated Meat Product Using an Engineered Yeast Expressing Cytoplasmic, Exogenous Bovine Collagen

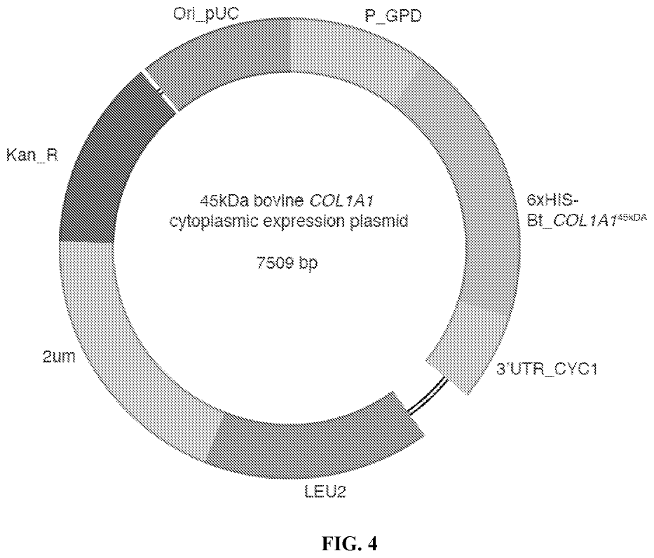

Cytoplasmic expression plasmid design. A 45 kDa fragment of the bovine COL1A1 gene was cloned into a Saccharomyces cerevisiae cytoplasmic expression plasmid containing LEU2 auxotrophic selection marker, an ampicillin resistance marker, and a 2 um origin. COL1A1 expression was driven from the GPD promoter. The resulting plasmid is shown in FIG. 4.

Yeast strain generation. Saccharomyces cerevisiae strain s288c (MATα SUC2 gal2 mal2 mel flo1 flo8-1 hap1 ho bio1 bio6) (Mortimer R K, Johnston J R. Genealogy of principal strains of the yeast genetic stock center. Genetics. 1986 May;113(1):35-43. doi: 10.1093/genetics/113.1.35. PMID: 3519363; PMCID: PMC1202798.) was grown in liquid YPD medium (1% yeast extract, 2% peptone, 2% glucose) to OD600 of 0.4-0.8. The yeast was transformed with the cytoplasmic expression plasmid (FIG. 4) using the lithium acetate transformation method (Gietz R D, Schiestl R H. High-efficiency yeast transformation using the LiAc/SS carrier DNA/PEG method. Nat Protoc. 2007;2(1):31-4. doi: 10.1038/nprot.2007.13. PMID: 17401334.) and plated on synthetic complete leucine-dropout (SC-LEU) agar plates (0.67% yeast nitrogen base without amino acids, 2% glucose, synthetic drop-out media supplement without leucine, 2% agar). Auxotrophic transformants were picked and re-streaked onto fresh SC-LEU plates. Auxotrophic strains were maintained in/on selective media for all downstream applications.

Scaffold generation—for flat sheets. 5 mL liquid SC-LEU (0.67 yeast nitrogen base without amino acids, 2% glucose, synthetic drop-out media supplement without leucine) was inoculated with the above transformed yeast strain and grown at 30° C. with shaking for 16 hours. A. oryzae spores were grown on BFM plates (0.67% yeast nitrogen base, 0.5% gluconic acid, 2% agar) for 2-3 days. A. oryzae spores were harvested from plates with sterile water. An equimolar mixture of yeast and A. oryzae spores in sterile water was plated onto SC-LEU agar plate with a thin film of cellophane overlaid on top of the agar. The plate was incubated at 30° C. for 1-2 days until a finely fuzzy layer was visible on top of the overlaid cellophane. The mycelium-yeast symbiotic growth structure was then peeled off the cellophane layer and transferred to a sterile flask containing 50 mL SC-LEU and incubated at 30° C. without shaking for 16-24 hours. The flask was then transferred to a shaker and grown an additional 16-24 hours at 30° C. with gentle shaking. The mycelium-yeast symbiotic growth was then harvested by washing with an excess of sterile water and incubated with 40% ethanol for 90 minutes. The scaffolds were then stored in PBS. Flat scaffolds were cut into desired shapes using a sterile scalpel.

Scaffold generation—for spheres. 5 mL liquid SC-LEU (0.67 yeast nitrogen base without amino acids, 2% glucose, synthetic drop-out media supplement without leucine) was inoculated with the above transformed yeast strain and grown at 30° C. with shaking for 16 hours. A. oryzae spores were grown on BFM plates (0.67% yeast nitrogen base, 0.5% gluconic acid, 2% agar) for 2-3 days. A. oryzae spores were harvested from plates with sterile water. 5 mL SD-LEU was inoculated with an equimolar mixture of yeast and A. oryzae spores in sterile water and grown with vigorous shaking at 30° C. for 16-24 hours. The spherical mycelial-yeast symbiotic growth structures were then harvested by washing with an excess of sterile water and incubated with 40% ethanol for 90 minutes. The spherical scaffolds were then stored in PBS.

Cell seeding and growth. Flat scaffolds were cut into 35 mm diameter circles and transferred to the wells of a 6-well tissue culture plate. Scaffolds were washed with 3 mL growth media (GM: DMEM high glucose, no pyruvate+10% FBS+1% pen/strep). A 3 mL suspension of QM7 cells at 4×10\hat{ }5 cells/mL in GM were then added dropwise to the scaffolds in the tissue culture well. Cells on scaffolds were incubated in a humidified incubator at 37° C. with 5% CO₂. 3 mL fresh GM was replaced every other day for 2-3 weeks. A cell-laden scaffold was sacrificed at day two to assay for cell adhesion via fluorescence microscopy.

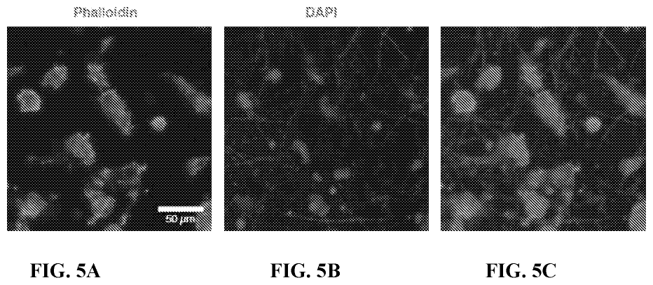

Fluorescence confocal microscopy. A cell-laden scaffold was transferred to a fresh 35 mm petri dish. Cells were fixed with 1 mL 4% paraformaldehyde for 20 minutes at room temperature. The cell-laden scaffold was then incubated in PBS+0.2% triton X-100 for 15 minutes at room temperature. The cell-laden scaffold was then incubated in a primary antibody solution of 543-phalloidin and DAPI in PBS for 30-60 minutes at room temperature. The cell-laden scaffold was washed twice in PBS with a 15 minute incubation at room temperature. The stained cell-laden scaffold was then transferred to a microscope slide, sandwiched with a coverglass, and then imaged by fluorescence confocal microscopy to confirm animal cell adhesion to the scaffold. As shown in FIGS. 5A-5C, the resulting scaffold was covered in actin networks (FIG. 5A) and cells possessing nuclei (FIG. 5B), with the merged view (FIG. 5C) demonstrating an intact cell-laden scaffold.

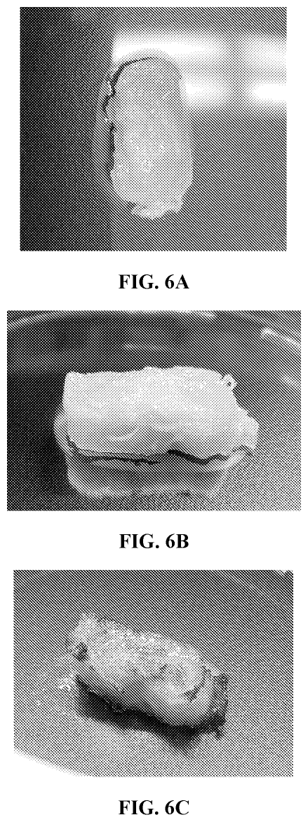

Assembly of cultivated meat product. QM7 cell-laden scaffolds were washed extensively with sterile PBS at harvest. The cell-laden scaffolds were dusted with a casein+microbial transglutaminase powder mixture (MooGloo RM). The dusted scaffolds were then layered on top of one another into a three-dimensional shape. The mass was then incubated at 37° C. for 16 hours to allow the transglutaminase to “glue” the pieces together (FIG. 6A, top view, FIG. 6B, side view). The cultivated quail meat product was cooked in a pan with avocado oil (FIG. 6C).

Results: As shown in FIGS. 6A-6C, this experiment resulted in the successful generation of a cultivated meat product with an enhanced uniformity of structure as compared to the meat product of FIGS. 3A-3B.

Accordingly, these results demonstrate that the methods of the present technology are useful for generating cell-based meat products comprising an edible biomaterial scaffold comprising a filamentous fungus, a plurality of engineered yeast cells expressing an exogenous protein, such as collagen, targeted to a subcellular location, and a plurality of one or more cell types, such as myoblasts.

### Example 9: Generation of Structured Cultivated Meat Product Using an Engineered Yeast Expressing Secreted, Exogenous Bovine Collagen

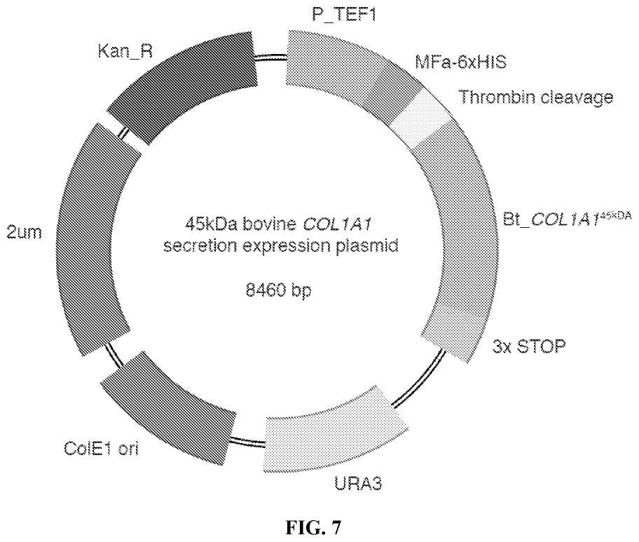

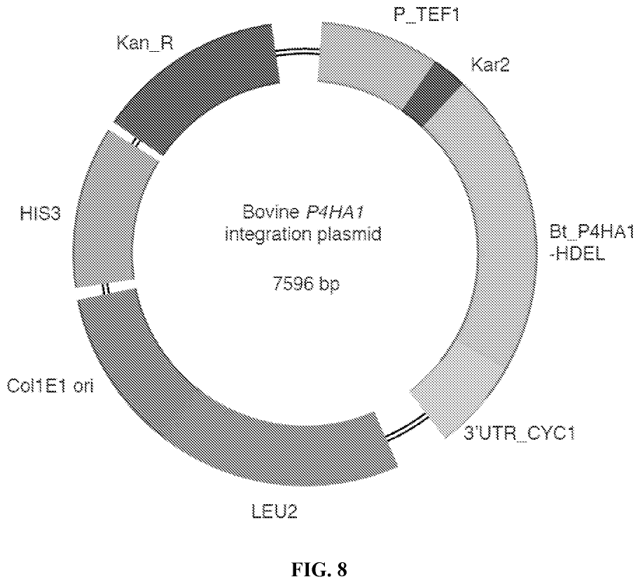

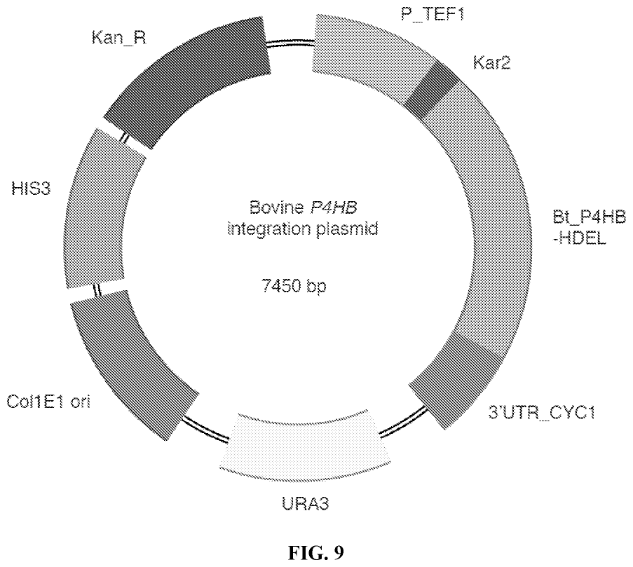

Secretion and Integration expression plasmid designs. A 45 kDa fragment of the bovine COL1A1 gene was cloned into a Saccharomyces cerevisiae secretion expression plasmid containing a URA3 auxotrophic selection marker, a kanamycin resistance marker, a 2 um element for S. cerevisiae propagation, and a ColE1 origin for E. coli propagation. A Mating Factor alpha (MFa) leader sequence with a 3′ thrombin cleavage site was fused to the 5′ end of the 45 kDa fragment of the bovine COL1A1 gene followed by a 3× stop. Expression of the MFa-COL1A1^{45 kDa} fusion was driven from the TEF1 promoter. The resulting secretion expression plasmid is shown in FIG. 7. The bovine P4HA1 gene was cloned into a Saccharomyces cerevisiae integration plasmid containing a dual LEU2 and HIS3 auxotrophic selection marker, a kanamycin resistance marker, and a ColE1 origin of replication for E. coli propagation. The P4HA1 gene was fused to the 3′ end of a Kar2 leader sequence and an HDEL 3′ terminal peptide sequence for targeting to the endoplasmic reticulum. The resulting P4HA1-integration plasmid is shown in FIG. 8. The bovine P4HB gene was cloned into a Saccharomyces cerevisiae integration plasmid containing a dual URA3 and HIS3 auxotrophic selection marker, a kanamycin resistance marker, and a ColE1 origin of replication for E. coli propagation. The P4HB gene was fused to the 3′ end of a Kar2 leader sequence and an HDEL 3′ terminal peptide sequence for targeting to the endoplasmic reticulum. The resulting P4HB-integration plasmid is shown in FIG. 9.

Yeast strain generation. A serial transformation procedure was performed to generate a triple-transformant containing genome-integrated P4HA1 and P4HB, and the MFa-COL1A1 plasmid maintained under auxotrophic selection. Saccharomyces cerevisiae strain s288c (MATα SUC2 gal2 mal2 mel flo1 flo8-1 hap1 ho bio1 bio6) (Mortimer R K, Johnston J R. Genealogy of principal strains of the yeast genetic stock center. Genetics. 1986 May;113(1):35-43. doi: 10.1093/genetics/113.1.35. PMID: 3519363; PMCID: PMC1202798.) was grown in liquid YPD medium (1% yeast extract, 2% peptone, 2% glucose) to OD600=0.4-0.8. The yeast were then transformed with the P4HB-integration plasmid containing Kar2-P4HB-HDEL using the lithium acetate transformation method (Gietz R D, Schiestl R H. High-efficiency yeast transformation using the LiAc/SS carrier DNA/PEG method. Nat Protoc. 2007;2(1):31-4. doi: 10.1038/nprot.2007.13. PMID: 17401334.) and plated on synthetic complete uracil-dropout (SC-URA) agar plates (0.67% yeast nitrogen base without amino acids, 2% glucose, synthetic drop-out media supplement without leucine, 2% agar). Uracil auxotrophic transformants were picked and re-streaked onto fresh SC-URA plates, then restreaked again onto non-selective rich media YPD agar (1% yeast extract, 2% peptone, 2% glucose, 2% agar). The single transformant was then grown overnight in liquid YPD and transformed with the P4HA1-integration plasmid using the lithium acetate method and plated onto SC-LEU plates. Transformants were streaked onto SC-LEU plates, then re-streaked onto YPD plates. The P4HA1 and P4HB double transformant was then grown in YPD and transformed with the MFa-COL1A1 secretion plasmid using the lithium acetate transformation method and plated onto SC-HIS plates. Transformants were picked and streaked onto SC-HIS plates. The final auxotrophic strain was maintained in selective dropout-HIS media for all downstream applications.

Making scaffolds with secreted COL1A1. 5 mL liquid SC-HIS (0.67 yeast nitrogen base without amino acids, 2% glucose, synthetic drop-out media supplement without histidine) was inoculated with the triple transformant yeast strain and grown at 30° C. with shaking for 16 hours. A. oryzae spores were grown BFM plates (0.67% yeast nitrogen base, 0.5% gluconic acid, 2% agar) for 2-3 days. A. oryzae spores were harvested from plates with sterile water. An equimolar mixture of yeast and A. oryzae spores in sterile water was plated onto SC-HIS agar plate with a thin film of cellophane overlaid on top of the agar. The plate was incubated at 30° C. for 1-2 days until a finely fuzzy layer was visible on top of the overlaid cellophane. The mycelium-yeast symbiotic growth structure was then peeled off the cellophane layer and transferred to a sterile flask containing 50 mL SC-HIS and incubated at 30° C. without shaking for 16-24 hours. The flask was then transferred to a shaker and grown an additional 16-24 hours at 30° C. with gentle shaking. The mycelium-yeast symbiotic growth was then harvested by washing with an excess of sterile water and incubated with 40% ethanol for 90 minutes. The scaffolds were then stored in PBS. Flat scaffolds were cut into desired shapes using a sterile scalpel (data not shown).

Accordingly, these results demonstrate that the methods of the present technology are useful for generating an edible biomaterial scaffold comprising a filamentous fungus and a plurality of engineered yeast cells.

### Example 10: Assembly of Cultivated Meat Product Using Secreted-Collagen Scaffolds

Cell seeding and growth. Flat scaffolds generated in Example 9 are cut into 35 mm diameter circles and transferred to the wells of a 6-well tissue culture plate. Scaffolds are washed with 3 mL growth media (GM: DMEM high glucose, no pyruvate+10% FBS+1% pen/strep). A 3 mL suspension of QM7 cells at 4×10\hat{ }5 cells/mL in GM are added dropwise to the scaffolds in the tissue culture well. Cells on scaffolds are incubated in a humidified incubator at 37° C. with 5% CO₂. 3 mL fresh GM is replaced every other day for 2-3 weeks.

Assembly of cultivated meat product. QM7 cell-laden scaffolds are washed extensively with sterile PBS at harvest. The cell-laden scaffolds are dusted with a casein+microbial transglutaminase powder mixture (MooGloo RM). The dusted scaffolds are layered on top of one another into a three-dimensional shape. The mass is then incubated at 37° C. for 16 hours to allow the transglutaminase to “glue” the pieces together. The cultivated quail meat product is cooked in a pan with avocado oil.

Results: It is anticipated that these experiments will result in the successful generation of a cultivated meat product.

Accordingly, these results will demonstrate that the methods of the present technology are useful for generating cell-based meat products comprising an edible biomaterial scaffold comprising a filamentous fungus, a plurality of engineered yeast cells expressing a secreted exogenous protein, such as collagen, and a plurality of one or more cell types, such as myoblasts.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

What is claimed is:

1. A biocapsule for use in production of a cell-based meat product, wherein the biocapsule comprises a biomaterial scaffold comprising:
(i) a filamentous fungus, and
(ii) a plurality of yeast cells.

2. The biocapsule of claim 1, wherein the filamentous fungus comprises mycelia.

3. The biocapsule of claim 1 or claim 2, wherein the plurality of yeast cells are genetically engineered to express one or more heterologous proteins.

4. The biocapsule of claim 3, wherein the one or more heterologous proteins are selected from one or more extracellular matrix (ECM) proteins, growth factor proteins, cell adhesion proteins, cell surface proteins, cell signaling proteins, protein motifs (synthetic or naturally-occurring), flavor proteins, or any combination thereof.

5. The biocapsule of any one of claims 1-4, wherein the filamentous fungus comprises one or more strains belonging to a division selected from the group consisting of Ascomycota, Basidiomycota, and Zygomycota (Mucoromycota and Zoopagomycota).

6. The biocapsule of claim 5, wherein the filamentous fungus strain is from the Ascomycota division and comprises one or more strains belonging to a genus selected from the group consisting of Aspergillus, Penicillium, and Neurospora.

7. The biocapsule of claim 6, wherein the filamentous fungus is from the Aspergillus genus and comprises one or more strains of Aspergillus oryzae.

8. The biocapsule of claim 6, wherein the filamentous fungus is from the Neurospora genus and comprises one or more strains of Neurospora crassa.

9. The biocapsule of claim 5, wherein the filamentous fungus strain is a strain of Rhizopus oryzae or Rhizopus oligosporus from the Mucoromycota division.

10. The biocapsule of any one of claims 1-9, wherein the plurality of yeast cells comprise one or more strains selected from the group consisting of Saccharomyces cerevisiae, Saccharomyces bayanous, Saccharomyces capensis, Schizosaccharomyces pombe, and Pichia pastoris.

11. The biocapsule of claim 10, wherein the plurality of yeast cells comprise one or more strains of Saccharomyces cerevisiae.

12. The biocapsule of claim 3, wherein the one or more heterologous proteins is a chordate ECM protein, growth factor protein, cell adhesion protein, cell signaling protein, protein motif (synthetic or naturally-occurring), flavor protein, cell surface protein, or any combination thereof.

13. The biocapsule of claim 12, wherein the heterologous chordate protein is a mammalian ECM protein, growth factor protein, adhesion molecule protein, cell signaling protein, protein motif (synthetic or naturally-occurring), flavor protein, cell surface protein, or any combination thereof.

14. The biocapsule of claim 3, wherein the one or more heterologous proteins comprise one or more ECM proteins selected from the group consisting of: collagen, laminin, fibronectin, and RGD motif peptides.

15. The biocapsule of claim 3, wherein the one or more heterologous proteins are expressed solubly, targeted to a specific subcellular localization, or secreted.

16. The biocapsule of any one of claims 1-15, wherein the plurality of yeast cells are fixed or decellularized.

17. A cell-based meat product comprising:
(i) an edible biomaterial scaffold comprising:
(a) a filamentous fungus, and
(b) a plurality of yeast cells; and
(ii) a plurality of one or more cell types.

18. The cell-based meat product of claim 17, wherein the plurality of one or more cells types comprises one or more cell types selected from the group consisting of: myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, embryonic stem cells, mesenchymal stem cells, and any combination thereof.

19. The cell-based meat product of claim 17 or claim 18, wherein the plurality of yeast cells are genetically engineered to express one or more heterologous proteins.

20. The cell-based meat product of any one of claims 17-19, wherein the plurality of one or more cell types is a chordate cell.

21. The cell-based meat product of claim 20, wherein the chordate cell is a mammalian cell.

22. The cell-based meat product of claim 20, wherein the chordate cell is a porcine cell, a bovine cell, or a poultry cell.

23. The cell-based meat product of any one of claims 17-22, wherein the filamentous fungus comprises mycelia.

24. The cell-based meat product of claim 19, wherein the one or more heterologous proteins are selected from one or more extracellular matrix (ECM) proteins, growth factor proteins, cell adhesion proteins, cell surface proteins, cell signaling proteins, protein motifs (synthetic or naturally-occurring), flavor proteins, or any combination thereof.

25. The cell-based meat product of any one of claims 17-24, wherein the filamentous fungus comprises one or more strains belonging to a division selected from the group consisting of Ascomycota, Basidiomycota, and Zygomycota (Mucoromycota and Zoopagomycota).

26. The cell-based meat product of claim 25, wherein the filamentous fungus strain is from the Ascomycota division and comprises one or more strains belonging to a genus selected from the group consisting of Aspergillus, Penicillium, and Neurospora.

27. The cell-based meat product of claim 26, wherein the filamentous fungus is from the Aspergillus genus and comprises one or more strains of Aspergillus oryzae.

28. The cell-based meat product of claim 26, wherein the filamentous fungus is from the Neurospora genus and comprises one or more strains of Neurospora crassa.

29. The cell-based meat product of claim 25, wherein the filamentous fungus strain is a strain of Rhizopus oryzae or Rhizopus oligosporus from the Mucoromycota division.

30. The cell-based meat product of any one of claims 17-29, wherein the plurality of yeast cells comprise one or more strains selected from the group consisting of Saccharomyces cerevisiae, Saccharomyces bayanous, Saccharomyces capensis, Schizosaccharomyces pombe, and Pichia pastoris.

31. The cell-based meat product of claim 30, wherein the plurality of yeast cells comprise one or more strains of Saccharomyces cerevisiae.

32. The cell-based meat product of claim 19, wherein the one or more heterologous proteins is a chordate ECM protein, growth factor protein, cell adhesion protein, cell signaling protein, protein motif (synthetic or naturally-occurring), flavor protein, or cell surface protein.

33. The cell-based meat product of claim 32, wherein the heterologous chordate protein is a mammalian ECM protein, growth factor protein, adhesion molecule protein, cell signaling protein, protein motif (synthetic or naturally-occurring), flavor protein, or cell surface protein.

34. The cell-based meat product claim 19, wherein the one or more heterologous proteins comprise one or more ECM proteins selected from the group consisting of: collagen, laminin, fibronectin, and RGD motif peptides.

35. The cell-based meat product claim 19, wherein the one or more heterologous proteins are expressed solubly, targeted to a specific subcellular localization, or secreted.

36. The cell-based meat product of any one of claims 17-35, wherein the plurality of yeast cells are fixed or decellularized.

37. A method for producing a filamentous fungus-yeast biocapsule comprising:
adding to a filamentous fungus culture comprising mycelia a plurality of yeast cells to form a filamentous fungus-yeast preparation, and
culturing the preparation under conditions to induce spontaneous co-immobilization of the filamentous fungus and yeast to form a filamentous fungus-yeast biocapsule.

38. The method of claim 37, wherein the conditions to induce co-immobilization of the filamentous fungus and yeast comprises the use of a first culture medium, and a second culture medium.

39. The method of claim 37 or claim 38, wherein the plurality of yeast cells are genetically engineered to express one or more heterologous proteins.

40. The method of claim 38 wherein the first culture medium comprises a carbon source usable by the filamentous fungus and not usable by the yeast, and wherein the carbon source is selected from the group consisting of: gluconic acid, starch, cellulose, inulin, and other similar colloidal molecules.

41. The method of claim 38 wherein the second culture medium comprises a preferred carbon source for the yeast that is also usable by the filamentous fungus.

42. The method of claim 37, further comprising contacting the filamentous fungus with an enzyme to modify one or more of the density, porosity, or texture of the filamentous fungus.

43. The method of claim 42, wherein the enzyme is selected from the group consisting of: zymolase, lyticase, and glucalase.

44. The method of any one of claims 37-43, further comprising fixing or decellularizing the yeast cells.

45. The method of any one of claims 37-44, wherein the filamentous fungus-yeast biocapsule is used as an edible biomaterial scaffold for cell-based meat production.

46. The method of any one of claims 37-45, further comprising shaping the filamentous fungus-yeast biocapsule.

47. The method of claim 46, wherein the shape of the filamentous fungus-yeast biocapsule resembles the shape of a three-dimensional meat product.

48. The method of claim 37, further comprising:
seeding the filamentous fungus-yeast biocapsule with a plurality of one or more cell types selected from the group consisting of: myoblasts or progenitor cells thereof, adipocytes or progenitor cells thereof, fibroblasts or progenitor cells thereof, endothelial cells or progenitor cells thereof, smooth muscle cells or progenitor cells thereof, myosatellite cells, induced pluripotent stem cells, embryonic stem cells, mesenchymal stem cells, and any combination thereof, to form a seeded biocapsule; and
culturing the seeded biocapsule under conditions effective to induce differentiation of the plurality of one or more cell types to produce an edible cell-based meat product.

49. The method of claim 48, wherein the plurality of one or more cell types is a chordate cell.

50. The method of claim 49, wherein the chordate cell is a mammalian cell.

51. The method of claim 49, wherein the chordate cell is a porcine cell, a bovine cell, or a poultry cell.

52. The method claim 39, wherein the one or more heterologous proteins are selected from one or more extracellular matrix (ECM) proteins, growth factor proteins, cell adhesion proteins, cell surface proteins, cell signaling proteins, protein motifs (synthetic or naturally-occurring), flavor proteins, or any combination thereof.

53. The method of any one of claims 37-52, wherein the filamentous fungus comprises one or more strains belonging to a division selected from the group consisting of Ascomycota, Basidiomycota, and Zygomycota (Mucoromycota and Zoopagomycota).

54. The method of claim 53, wherein the filamentous fungus strain is from the Ascomycota division and comprises one or more strains belonging to a genus selected from the group consisting of Aspergillus, Penicillium, and Neurospora.

55. The method of claim 54, wherein the filamentous fungus is from the Aspergillus genus and comprises one or more strains of Aspergillus oryzae.

56. The method of claim 54, wherein the filamentous fungus is from the Neurospora genus and comprises one or more strains of Neurospora crassa.

57. The method of claim 53, wherein the filamentous fungus strain is a strain of Rhizopus oryzae or Rhizopus oligosporus from the Mucoromycota division.

58. The method of any one of claims 37-57, wherein the plurality of yeast cells comprise one or more strains selected from the group consisting of Saccharomyces cerevisiae, Saccharomyces bayanous, Saccharomyces capensis, Schizosaccharomyces pombe, and Pichia pastoris.

59. The method of claim 58, wherein the plurality of yeast cells comprise one or more strains of Saccharomyces cerevisiae.

60. The method of claim 39, wherein the one or more heterologous proteins is a chordate ECM protein, growth factor protein, cell adhesion protein, cell signaling protein, peptide motif (synthetic or naturally-occurring), flavor protein, or cell surface protein.

61. The method of claim 60, wherein the heterologous chordate protein is a mammalian ECM protein, growth factor protein, adhesion molecule protein, cell signaling protein, peptide motif (synthetic or naturally-occurring), flavor protein or cell surface protein.

62. The method claim 39, wherein the one or more heterologous proteins comprise one or more ECM proteins selected from the group consisting of: collagen, laminin, fibronectin, and RGD motif peptides.

63. The method of claim 39, wherein the one or more heterologous proteins are expressed solubly, targeted to a specific subcellular localization, or secreted.
